(19) United States
(12) Patent Application Publication
(10) Pub. No.: US 2015/0201661 A1
(43) Pub. Date: Jul. 23, 2015

(54) BAMBOO EXTRACTS, COMPOSITIONS AND USES THEREOF

(71) Applicants: Marvin Heuer, Orlando, FL (US); Ying Zhang, Hangzhou (CN); William Roberts, Orlando, FL (US); Luolian Huang, Hangzhou (CN)

(72) Inventors: Marvin Heuer, Orlando, FL (US); Ying Zhang, Hangzhou (CN); William Roberts, Orlando, FL (US); Luolian Huang, Hangzhou (CN)

(21) Appl. No.: 14/600,478

(22) Filed: Jan. 20, 2015

Related U.S. Application Data
(60) Provisional application No. 61/929,270, filed on Jan. 20, 2014.
(60) Provisional application No. 61/929,274, filed on Jan. 20, 2014.
(60) Provisional application No. 61/929,278, filed on Jan. 20, 2014.

(51) Int. Cl. A23L 1/30; A23L 1/29; A61K 36/899

(52) U.S. Cl. CPC A23L 1/3004; A61K 36/899; A23L 1/293; A23V 2002/00

(57) ABSTRACT

The present invention relates to methods and compositions for improving the health of humans and animals, including lowering body weight and reducing fat in a human or an animal, and for improving food quality of animals. More particularly, the present invention is a phytosterol-containing extract derived from bamboo that lowers body weight by reducing or inhibiting body weight and reducing fat. Methods of making and using such compositions are also provided.

### FIELD OF THE INVENTION

The present invention relates to methods and compositions for lowering body weight and reducing fat in an animal. More particularly, the present invention is a phytosterol-containing extract derived from bamboo that lowers body weight by reducing or inhibiting body weight and reducing fat. Methods of making and using such compositions are also provided. The extract preferably contains phytosterols, polyphenols, phenolic acid, and may preferably contain one or more active agents selected from the group consisting of flavonoids, p-coumaric acid, caffeic acid, ferulic acid and chlorogenic acid.

### BACKGROUND OF THE INVENTION

In traditional Chinese medicine, bamboo leaves are used as a component to reduce the energy of “fire” (an element usually related to inflammation), and treat hypertension, arteriosclerosis, and cardiovascular disease. Besides the medicinal application, bamboo leaves and stems have been traditionally used in family kitchens to enhance the flavor and color of food. Correlations between the use of bamboo components in cooking processes and generally improved health status of the residents have been observed, but solid scientific examinations are yet to be conducted. Common edible bamboo products are made out of the most nutritious parts of the bamboo, containing a complex source of amino acids, vitamins and minerals. It is currently marketed for those who have symptoms associated with upper respiratory problems such as cough with phlegm, fever, runny nose, sore throat, dry mouth, heaviness in the chest, and headaches. Moreover, for the purpose of benefiting health and preserving the products from oxidation, bamboo extracts are also used in the beverage and food industries.

Silica is marketed as an essential mineral for maintaining the integrity and health of the skin, ligaments, tendons and bones. Bamboo extracts are the richest known source of silica; containing over 70% organic silica. Silica is marketed as having a restorative effect on many of the body's tissues and bamboo extracts are sold for their silica content. Such extracts are purportedly to prevent premature aging and preserve skin youthfulness, support joint health, maintains vascular and heart health, support nervous and glandular system health, builds healthy bones, nails and teeth, prevents wrinkles and keeps skin beautiful, promote the growth of thick, strong hair, and radiant skin.

Bamboo belongs to the Poaceae (also called Gramineae or true grasses) family. There are about 280 known species of bamboo all over the world. More than 10 genera are divided into about 1,450 species. Bamboo species are found in diverse climates, from cold mountains to hot tropical regions. They also occur in sub-Saharan Africa, and in the Americas from the mid-Atlantic United States south to Argentina and Chile.

Bamboo grows in two main forms: the woody bamboos (Arundinarieae and Bambuseae) and the understory herbaceous bamboos (Olyreae). Molecular analysis suggests that there are 3-5 major lineages of bamboo. Four major lineages are currently recognized: temperate woody, paleotropical woody, neotropical woody and herbaceous. Phyllostachys ambusoides (Cedrela sinesis), Phyllostachys nigra and Phyllostachys edulis are cultivated.

World-wide the area of bamboo grove is approximately 20,000,000 hectare. China is one of main bamboo producing countries in the World with over 7,000,000 hectare of bamboo. There are over 50 species of bamboo in Phylloslachys Sieb. et Zucc and many of these species have been produced in China. The most commonly harvested bamboo is Phyllolachyspubescens, which represents a majority of World bamboo yields.

Traditionally, bamboo has been reported to be effective in treating palsy and hypertension, and was used to treat pneumonia and bronchitis to bring down fever, loosen phlegm and as a coolant. Recently, it has been reported that Bamboo has been used to treat hypertension, atherosclerosis and cardiovascular disease. See U.S. Pat. No. 7,897,182. Bamboo is also known to have anti-oxidant effect which is effective in the prevention of cancer and aging. Also, phytochemicals such as organic acids, tannin, benzofuran within the plant are expected to contribute to preventing diseases of the circulatory system.

A free radical can be defined as any molecular species capable of independent existence that contains an unpaired electron in an atomic orbital. The presence of an unpaired electron results in certain common properties that are shared by most radicals. Many radicals are unstable and highly reactive. They can either donate an electron to or accept an electron from other molecules, therefore behaving as oxidants or reductants. The most important oxygen-containing free radicals in many disease states are hydroxyl radical, superoxide anion radical, hydrogen peroxide, oxygen singlet, hypochlorite, nitric oxide radical, and peroxynitrite radical. These are highly reactive species, capable in the nucleus, and in the membranes of cells of damaging biologically relevant molecules such as DNA, proteins, carbohydrates, and lipids.

Oxidative stress and oxidative modification of biomolecules are involved in a number of physiological and pathophysiological processes such as aging, atherosclerosis, inflammation and carcinogenesis, and drug toxicity.

An antioxidant is a molecule stable enough to donate an electron to a rampaging free radical and neutralize it, thus reducing its capacity to damage. These antioxidants delay or inhibit cellular damage mainly through their free radical scavenging property. These low-molecular-weight antioxidants can safely interact with free radicals and terminate the chain reaction before vital molecules are damaged. Some of such antioxidants, including glutathione, ubiquinol, and uric acid, are produced during normal metabolism in the body. Other lighter antioxidants are found in the diet. Although there are several enzymes system within the body that scavenge free radicals, the principle micronutrient (vitamins) antioxidants are vitamin E (α-tocopherol), vitamin C (ascorbic acid), and B-carotene. The body cannot manufacture these micronutrients, so they must be supplied in the diet.

Two principle mechanisms of action have been proposed for antioxidants. The first is a chain-breaking mechanism by which the primary antioxidant donates an electron to the free radical present in the systems. The second mechanism involves removal of ROS/reactive nitrogen species initiators (secondary antioxidants) by quenching chain-initiating catalyst. Antioxidants may exert their effect on biological systems by different mechanisms including electron donation, metal ion chelation, co-antioxidants, or by gene expression regulation.

Synthetic and natural antioxidants are used routinely in foods and medicine especially those containing oils and fats to protect the food against oxidation. There are a number of synthetic phenolic antioxidants, butylated hydroxytoluene (BHT) and butylated hydroxyanisole (BHA) being prominent examples. These compounds have been widely uses as antioxidants in food industry, cosmetics, and therapeutic industry. However, some physical properties of BHT and BHA such as their high volatility and instability at elevated temperature, strict legislation on the use of synthetic food additives, carcinogenic nature of some synthetic antioxidants, and consumer preferences have shifted the attention of manufacturers from synthetic to natural antioxidants. In view of increasing risk factors of human to various deadly diseases, there has been a global trend toward the use of natural substance present in medicinal plants and dietary plats as therapeutic antioxidants. It has been reported that there is an inverse relationship between the dietary intake of antioxidant-rich food and medicinal plants and incidence of human diseases. The use of natural antioxidants in food, cosmetic, and therapeutic industry would be promising alternative for synthetic antioxidants in respect of low cost, highly compatible with dietary intake and no harmful effects inside the human body. Many antioxidant compounds, naturally occurring in plant sources have been identified as free radical or active oxygen scavengers. Attempts have been made to study the antioxidant potential of a wide variety of vegetables like potato, spinach, tomatoes, and legumes. There are several reports showing antioxidant potential of fruits. Strong antioxidants activities have been found in berries, cherries, citrus, prunes, and olives. Green and black teas have been extensively studied in the recent past for antioxidant properties since they contain up to 30% of the dry weight as phenolic compounds.

Medicinal plants also provide antioxidants and these include Acacia catechu (kair), Aegle marmelos (Bengal quince, Bel), Allium cepa (Onion), A. sativum (Garlic, Lahasuna), Aleo vera (Indian aloe, hritkumari), Amomum subulatum (Greater cardamom, Bari elachi), Andrographis paniculata (Kiryat), Asparagus recemosus (Shatavari), Azadirachta indica (Neem, Nimba), Bacopa monniera (Brahmi), Butea monosperma (Palas, Dhak), Camellia sinensis (Green tea), Cinnamomum verum (Cinnamon), Cinnamomum tamala (Tejpat), Curcma longa (Turmeric, Haridra), Emblica officinalis (Inhian gooseberry, Amlaki), Glycyrrhiza glapra (Yashtimudhu), Hemidesmus indicus (Indian Sarasparilla, Anantamul), Indigofera tinctoria, Mangifera indica (Mango, Amra), Momordica charantia (Bitter gourd), Murraya koenigii (Curry leaf), Nigella sativa (Black cumin), Ocimum sanctum (Holy basil, Tusil), Onosma echioides (Ratanjyot), Picrorrhiza kurroa (Katuka), Piper beetle, Plumbago zeylancia (Chitrak), Sesamum indicum, Sida cordifolia, Spirulina fusiformis (Alga), Swertia decursata, Syzigium cumini (Jamun), Terminalia ariuna (Arjun), Terminalia bellarica (Beheda), Tinospora cordifolia (Heart leaved moonseed, Guduchi), Trigonella foenum-graecium (Fenugreek), Withania somifera (Winter cherry, Ashwangandha), and Zingiber officinalis (Ginger).

The prevalence of obesity has significantly increased during the last decades reaching epidemic proportions in many countries. Obesity has been described as a state of chronic oxidative stress. Oxidative stress has been defined as the link between obesity and its major associated disorders such as insulin resistance, hypertension, etc. Recent studies have suggested the potential therapeutic role of dietary antioxidant supplementation in the reduction of body weight or its beneficial effect on several obesity related disorders.

Leptin binds to neuropeptide Y (NPY) neurons in the arcuate nucleus in such a way as to decrease the activity of these neurons. One of the roles of leptin is to signal the brain that the body has had enough to eat, producing a feeling of satiety. Leptin may make it easier for people to resist the temptation of foods high in calories.

Circulating leptin levels give the brain input regarding energy storage, so it can regulate appetite and metabolism. Leptin works by inhibiting the activity of neurons that contain neuropeptide Y (NPY) and agouti-related peptide (AgRP), and by increasing the activity of neurons expressing α-melanocyte-stimulating hormone (α-MSH). The NPY neurons are a key element in the regulation of appetite; small doses of NPY injected into the brains of experimental animals stimulates feeding, while selective destruction of the NPY neurons in mice causes them to become anorexic.

Adiponectin is a protein hormone that modulates a number of metabolic processes, including glucose regulation and fatty acid oxidation. Adiponectin is secreted from adipose tissue into the bloodstream. Levels of adiponectin are inversely correlated with body fat percentage in adult humans. Transgenic mice with increased adiponectin show impaired adipocyte differentiation and increased energy expenditure associated with protein uncoupling. Bauche IB, E I Mkadem S A, Pottier A M, Senou M, Many M C, Rersohazy R, Penicaud L, Maeda N, Funahashi T, Brichard SM (April 2007). “Overexpression of adiponectin targeted to adipose tissue in transgenic mice: impaired adipocyte differentiation”. Endocrinology 148 (4): 1539-49.

Adiponectin plays a role in the suppression of the metabolic derangements that may result in type 2 diabetes, obesity, atherosclerosis, non-alcoholic fatty liver disease (NAFLD) and an independent risk factor for metabolic syndrome. Adiponectin in combination with leptin has been shown to completely reverse insulin resistance in mice.

Weight reduction significantly increases circulating levels of adiponectin. Adiponectin exerts some of its weight reduction effects via the brain. This is similar to the action of leptin, but the two hormones perform complementary actions, and can have synergistic effects.

### SUMMARY OF THE INVENTION

The present invention is a composition for weight loss and reducing body weight and fat in an animal, including but not limited to a human. This composition includes a phytosterol-containing extract isolated from bamboo.

Another embodiment of the present invention is a dietary supplement that includes as an active agent a body weight and fat lowering amount of a phytosterol-containing extract isolated from bamboo.

Another embodiment of the present invention is a composition useful for increasing fat metabolism in an animal including but not limited to a human. The composition includes an effective amount of a phytosterol-containing extract isolated from bamboo.

A further embodiment of the invention is a pharmaceutically useful composition that includes an extract containing one or more phytosterols isolated from bamboo.

A further embodiment of the invention is a phytosterol-containing extract derived from bamboo that contains phytosterols, polyphenols, phenolic acid, flavonoids, p-coumaric acid, caffeic acid, ferulic acid and chlorogenic acid.

A further embodiment of the invention is a method for lowering body weight in an animal, including, but not limited to, a human. This method includes administering to the animal a composition that includes an effective amount of a phytosterol-containing extract isolated from bamboo sufficient to lower body weight and reduce fat in the animal.

In another embodiment, the invention is a method for increasing the muscle mass in an animal, including, but not limited to, a human. The method includes administering to the animal a composition that includes an effective amount of a phytosterol-containing extract isolated from bamboo, sufficient to increase muscle mass in the animal.

In another embodiment, the invention is a method of increasing the fat metabolism and/or decreasing the abdomenal fat of an animal, including but not limited to a human, by supplying an effective amount of a phytosterol-containing extract isolated from bamboo, sufficient to increase fat metabolism and/or decrease abdominal fat in the animal.

In a further embodiment, the invention comprises a method for treating an animal, including but not limited to, a human subject, suffering from a muscle-wasting or muscle weakness disease, such as sarcopenia or dynapenia. The method includes administering to the animal a composition that includes an effective amount of a phytosterol-containing extract isolated from bamboo, sufficient to reduce the effects of the muscle-wasting or muscle weakness disease.

In another embodiment, the invention comprises of a method for increasing muscular strength and/or endurance for an animal, including but not limited to, a human subject. The method includes administering to the animal a composition that includes an effective amount of a phytosterol-containing extract isolated form bamboo, sufficient to improve the muscle's cellular composition or increase concentration or improve structure of mitochondria in the animal.

A further embodiment of the invention comprises of a method for improving cellular composition, and energy by increasing mitochondrion concentration for an animal, including but not limited to, a human subject. The method includes administering to the animal a composition that includes an effective amount of a phytosterol-containing extract isolated form bamboo, sufficient to increase concentration and or improve structure of mitochondrion on a given tissue.

The present invention also includes a method of making a composition for lowering body weight in an animal, including but not limited to, a human. This method includes obtaining an extract of phytosterols from a source of bamboo and combining the extract with a suitable delivery vehicle for administering body weight-lowering amounts of the extract to the animal.

Another embodiment of the invention comprises composition for animal feed comprising a phytosterol-containing extract isolated from bamboo. The compositions can be used as improved feed stock for meat animals, such as poultry (chicken, turkey, duck, etc), livestock (beef and pork, etc) and fish and seafood products. In other embodiments, the invention comprises compositions for adding to animal feed compositions, comprising a phytosterol-containing extract isolated from bamboo. The compositions for addition to animal feed comprise a phytosterol-containing extract isolated from bamboo in an amount sufficient that, when added to an animal's normal daily diet ration, animal is improved in one or more characteristics selected from the group consisting of health, energy, and food quality. In particular embodiments, the methods comprise adding a composition of the invention to the daily diet of the animal.

Another embodiment of the invention comprises methods of improving the food quality of an animal. The methods comprise feeding the composition of composition of the invention to the animal, in an amount sufficient to improve the food quality, for example improving the sensory qualities of an animal, in at least one sensory quality selected from the group consisting of taste, appearance, smell and texture. In certain embodiments, the animal is selected from the group consisting of poultry, livestock and fish.

A method of improving endurance in a human subject, comprising administering an effective amount of a composition comprising a phytosterol-containing extract isolated from bamboo to the human subject, that is sufficient to improve the muscle's cellular composition.

A method of increasing improving cellular composition, and energy in a human subject comprising administering to the human subject a composition that includes an effective amount of a phytosterol-containing extract isolated form bamboo, that is sufficient to increase concentration and/or improve structure of mitochondria in the human subject.

### DESCRIPTION OF THE FIGURES

FIG. 1 shows the effects of a green bamboo extract of the invention on leptin and adiponectin levels in chicken.

FIG. 2 shows transmission electron microscopic images of back muscles of Micropterus salmoniodes. At a magnification of (15000×), A and B represent the mitochondria amounts observed in fish that were fed control (0 GBE) and 1.5 g/kg daily ration GBE, respectively.

FIG. 3 shows the effects of a green bamboo extract (GBE) of the invention on the body weight and fat content in different parts of large yellow croaker in three groups: Reference (or control) Group; GBE-supplemented Group; and Astaxanthin-supplemented Group (ASTA). Different small letters mean significant difference (p<0.05), different capital letters mean highly significant difference (p<0.01), the same letter or no letter means no significant difference (p>0.05).

FIG. 4 shows transmission electron microscopic images of back muscles of large yellow croakers in the three groups (a to c): a Reference group, b GBE group, c ASTA group (10000×); (A to C): A Reference group, B GBE group, C ASTA group (12000×).

### DETAILED DESCRIPTION OF THE INVENTION

The present invention includes a composition for reducing body weight and fat in an animal. This composition includes a phytosterol-containing extracts isolated from bamboo. As used herein, the term “phytosterol” includes the entire group of free phytosterols, phytosterol fatty acid esters and (acylated) phytosterol glucosides. Phytosterols include sitosterol, campesterol and stigmasterol, and in certain embodiments, these compounds will comprise about 65%:30%:3% of the extract total phytosterol contents, respectively. As used herein, the term ‘phytosterols’ may also include phytosterols in the form of saturated derivatives, known as stanols, and as esterified stanol esters. See Lichtenstein et al. (2001), Circulation, 103:1177-1180. As used herein, the term ‘derivatives of phytosterols’ includes esters, acids and salts of phytosterols, and may include other chemical modifications to phytosterols, such as those that occur naturally in the plant.

Extracts of the invention may have a total phenolic content of 5-30 percent, a polysaccharide content of 5-15 percent and a triterpene content of 3-10 percent. The extract has multiple functions as an antioxidant, a nutrition enhancer, an immunopotentiator, a lipid metabolism regulator, an animal product quality improver, a flavoring agent, a preservative, a feed attractant and the like.

The functional components of bamboo-leaf extracts are mainly flavone C-glycosides that are separately orientin, homoorientin, vitexin and isovitexin. C-glycoside flavones have several key benefits over oxygen flavonoid glycosides: structural stability and not easily degradable; and increased hydrophilicity which is advantageous to the development of foods, drugs and cosmetics. Toxicity studies have shown that the LD₅₀ of Bamboo extract is above 20 g/kg of body weight, meaning it is very non-toxic; there was no fatality or side effects at even 500 times the normal dosage given to mice.

Extracts of bamboo according to the invention may provide yellow, brown or pale yellow powder or granular formulations with a bamboo aroma and a total phenol content of 8% to 30% polysaccharide content of 5% to 15%, triterpenes content of 3% to 10% of the mass %.

In a preferred embodiment, the bamboo extract of this invention is a natural extract from the leaves of Phyllostachys Sieb. Et Zucc. from Bambusoideae family in Gramineae. A preferred method of bamboo leaf extraction was disclosed previously by applicants (CN patent No. ZL 98104563.4 and ZL 98104564.2; ZL 02154401.8 and ZL 200410099219.8).

The appearance of the bamboo leaf extract is a yellow or brown powder. The main components of the bamboo leaf extract include flavonoids (i.e. homoorientin, orientin, isovitexin and vitexin) and phenolic acids (i.e. chlorogenic acid, ferulic acid and caffeic acid). The total flavonoid content of the bamboo leaf extract is about 4%-50% and the total phenolic acid content is 10%-80%.

Numerous studies show that bamboo flavonoids have excellent anti-free radical, anti-oxidant, anti-aging, anti-bacterial, anti-viral effects and protect the heart and brain arteries, prevents senile degenerative disease and other biological effects. With its rich source of raw materials, explicit function components, convincing safety, high effective and stable formulation quality and freshly sweet bamboo fragrance, it has many benefits as a functional food, dietary supplement and cosmetic.

The latest research also demonstrates that bamboo flavonoids do not irritate the skin and mucosa, and has no allergic reactions; it is comparable with tea polyphenols and gingko leaf extracts as an active anti-free radical, anti-oxidant and anti-radiation substance; it also has significant inhibitive and anti-inflammatory effect; within the dosage range of about 0.005% to about 0.05%, it can significantly enhance the proliferation of skin cells, and inhibit melanin synthesis; within the dosage range of 0.0005% to about 0.005%, it can reduce the generation of MDA to a large extent, thereby raising SOD activity. It possesses sufficient and necessary pre-requisites to be a safe, highly effective and economical phytochemical anti-aging and skin care component.

The active agent or agents in the present compositions may also be derived from a crude extract of bamboo. The crude extract can been fractionated and analyzed for the presence of phytosterols. In particular, several phytosterols have been identified in various fractions of the crude extract using conventional analytical techniques, such as for example, gas chromatography-mass spectrometry (GC-MS) and liquid chromatography-mass spectrometry (LC-MS). Each fraction of the crude extract contains one or more phytosterols, and may include, for example, one or more of sitosterol, campesterol and stigmasterol.

The present invention also encompasses extracts derived from raw bamboo shoots, bamboo outer skin, and bamboo shell. Bamboo processing byproducts, namely green bamboo processing scraping scraps, bamboo powder or bamboo shavings are all useful in the invention.

In one embodiment, the extract is a novel extract that contains bamboo leaf polyphenol, bamboo shaving polysaccharides and bamboo shoot peptides, using Phyllostachys bamboo as raw material and involves the extraction of active ingredients from different parts of the bamboo by using advanced technologies such as adsorption-desorption and membrane separation. The bamboo secondary metabolites in the extract, which have antioxidant capacity, lipid metabolic regulation capacity and immunological enhancement capacity, include flavonoids, phenolic acids, polysaccharides, terpenoids, lactones, phytosterols, anthraquinone, amino acids, peptides, and mineral elements. This embodiment of the invention comprises a tan powder with a total phenol content of about 14%, polysaccharide content of about 11% and a total triterpenoid content of about 4%.

Useful extracts of the present compositions contain a mixture of phytosterols including, for example, sitosterol, sitastanol, stigmasterol and derivatives and isomers thereof. The extracts of the present invention can also include, for example, beta-sitosterol, stigmasta-3,5-dien-7 one, stigmast-4-en-3-one, stigmasta-5,22-dien-3-ol, campesterol, and derivatives and isomers thereof. For purposes of the present invention, the term “derivatives” is intended to encompass all chemically modified versions of the enumerated phytosterols which alone or in combination have a body weight lowering effect when administered to an animal. For example, chemically modified forms of phytosterols that are useful in the present invention include esterified, glycosidic, saturated or unsaturated and oxysterol forms thereof.

The present compositions include phytosterols derived from bamboo obtained from a variety of bamboo species including, for example, Bambusa oldhami Nakai, Bambusa edulis, Pseudosas usawai, Zizania latiflia, Saccharum officinarum, Dendrocalamus latiflorus Munro, Phyllostachys edulis, Phyllostachys pubescens, Phyllostachys nigra and Phyllostachys makinoi.

At least 17 phytosterols have been identified in bamboo extracts. The phytosterols in the crude extract are structurally diverse. For example, beta-sitosterol and campesterol are common sterols in plant leaves. Two saturated phytosterols are found in crude extracts which are believed to be isomers of sitostanol.

In the present invention, compositions which lower body weight in mammals can be formed from phytosterols extracted from bamboo. These compositions include a therapeutically effective amount of the crude phytosterol extract and a pharmaceutically or nutritionally acceptable carrier or excipient. Such a carrier includes but is not limited to saline, buffered saline, dextrose, water, glycerol, ethanol and combinations thereof. The exact formulation, of course, will suit the mode of administration.

In one embodiment the compositions of the invention may be pharmaceutical compositions. The pharmaceutical compositions of the present invention, if desired, can also contain minor amounts of wetting or emulsifying agents or pH buffering agents. These compositions can take various forms including, for example, solutions, suspensions, emulsions, tablets, pills, capsules, sustained release formulations or powders. These compositions can be formulated as a suppository with traditional binders and carriers, such as triglycerides. Oral formulations are also contemplated and can include standard carriers, such as pharmaceutical grades of mannitol, lactose, starch, magnesium stearate, sodium saccharine, cellulose, magnesium carbonate, etc.

The compositions of the present invention can be formulated as neutral or salt forms or derivatives of phytosterols. For purposes of the present invention, the term “derivatives” is intended to encompass all chemically modified versions of the enumerated phytosterols which alone or in combination have a body weight lowering effect when administered to an animal. For example, chemically modified forms of phytosterols that are useful in the present invention include esterified, glycosidic, saturated or unsaturated and oxysterol forms thereof.

Pharmaceutically and nutritionally acceptable salts include those formed with free amino groups, such as those derived from hydrochloric, phosphoric, acetic, oxalic, tartaric acids, etc. and those formed with free carboxyl groups such as those derived from sodium, potassium, ammonium, calcium, ferric hydroxides, isopropylamine, triethylamine, 2-ethylamino ethanol, histidine, procaine, etc. Those skilled in the art will be able to develop numerous other possible salts.

Doses of purified compositions useful for the invention are generally about 5-500 micrograms of active compound per kilogram body weight. Effective doses may be extrapolated from dose response curves derived from in vitro or animal model test systems. A skilled clinician or veterinarian will be able to develop and modify dosage regimens using standard practices, for example, titration of doses to optimize effects with minimal undesired or adverse effects.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate or mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

Pharmaceutical formulations containing present composition may be prepared in any oral dosage form such as powder, tablet, capsule, soft capsule, aqueous medicine, syrup, elixirs pill, powder, sachet, granule.

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or compound varies depending on the condition and the weight of the subject, severity, form, route and a period of administration, and may be chosen by those skilled in the art. When supplied as an additive to animal feeds, the extract is generally added in an amount ranging up to 10 g/kg of feed, preferably, about 0.1 to about 3 g/kg feed. For pharmaceutical and veterinary purposes of treating a condition, in order to obtain desirable effects, it is generally recommended to administer at the amount ranging up to 10 g/kg of body weight preferably, about 0.1 to 3 g/kg/day of the inventive extract or compounds of the present invention. The dose may be administered in single or divided into several times per day. In terms of composition, the amount of inventive extract should be present between 0.01 to 100% by weight.

The present compositions can be incorporated into dietary supplements and health foods. Such supplements include as an active ingredient effective amounts of the present composition to lower body weight in an animal, including humans. The formulation of dietary supplements is well known in the art and can include a suitable carrier, as well as minor amounts of a variety of materials including for example, wetting or emulsifying agents or pH buffering agents.

The specific formulation of the dietary supplements of the present invention will vary depending upon a number of factors, including the sex and weight of the patient, as well as the severity of the disease. The dietary supplements of the present invention, however, must include a sufficient amount of crude phytosterol extracts from bamboo to lower body weight in a patient. In particular, the extract in the supplement must be sufficient in amount to lower total body weight.

The dietary supplements of the present invention may include crude extracts that contain one or more phytosterols derived from bamboo. These extracts can include, for example, sitosterol, sitastanol, stigmasterol and derivatives and isomers thereof.

Above described composition therein can be added to food, additive or beverage, wherein the amount of above described extract in food or beverage may generally range from about 0.1 to 100 w/w %, preferably 1 to 50 w/w % of total weight of food for the health care food composition and 1 to 30 g, preferably 0.1 to 10 g per 100 ml of the health beverage composition.

Providing that the health beverage composition of present invention contains above described extract as an essential component in the indicated ratio, there is no particular limitation on the other liquid component, wherein the other component can be various deodorant or natural carbohydrate etc., such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol, etc. As the other deodorant than aforementioned ones, natural deodorants such as taumatin, stevia extract such as levaudioside A, glycyrrhizin etc., and synthetic deodorant such as saccharin, aspartame et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. Dietary use of the extracts may be various foods, beverages, gums and the like.

The inventive composition may additionally comprise one or more than one of organic acid, such as citric acid, fumaric acid, adipic acid, lactic acid, malic acid; phosphate, such as phosphate, sodium phosphate, potassium phosphate, acid pyrophosphate, polyphosphate; natural anti-oxidants, such as polyphenol, catechin, alpha-tocopherol, rosemary extract, vitamin C, green tea extract, licorice root extract, chitosan, tannic acid, phytic acid etc.

The above extract of bamboo plant may be 1 to 99% high concentrated liquid, power, or granule type.

Similarly, the above extract of bamboo plant can comprise additionally one or more than one of lactose, casein, dextrose, glucose, sucrose and sorbitol.

The present invention also includes a method for lowering body weight in an animal by administering to the mammal an effective amount of a phytosterol-containing extract isolated from bamboo sufficient to lower body weight. As used herein, the term animal includes mammals, fowl, and fish. The term “mammal” includes humans, as well as other species.

Another embodiment of the present invention includes a method of making the compositions for lowering body weight in mammals as set forth above. This-method includes obtaining an extract from a source of bamboo that contains a mixture of phytosterols and combining that extract with a suitable delivery vehicle for administering body weight-lowering amounts of the extract to an animal. The delivery vehicle can be any physiologically appropriate carrier for administering the body weight lowering extracts of the present invention to an animal. These delivery vehicles have been described in detail above and include both pharmaceutical preparations, as well as dietary supplements.

The bamboo extracts of the invention may be a mixture of plant sterols, oxygenated sterols and their ketone and aldehyde metabolites. The crude bamboo extracts (with the polar (water fraction) and both methanol soluble fractions and methylene chloride soluble fractions) are useful compositions of the invention as a result of the broad spectrum of phytosterols contained therein.

One of the chemical differences between the crude bamboo extract and the methanol soluble fraction/methylene chloride soluble fraction was the presence of ester forms of the sterol in the methanol soluble fraction. In the methylene chloride soluble fraction, phytosterols exist in the hydrophobic form. In the methanol soluble fraction, part of the sterols likely exist as an ester form and part of the sterols (methanol soluble fraction) exist as a straight hydrophobic form.

The bamboo extracts of the present invention can be prepared by extracting bamboo with water, organic solvent, or mixing solvents thereof. Conventional solvents can be used as organic solvents, polar solvents such as water, C₁₋₄ alcohol (such as methanol, ethanol etc.), etc., or mixtures of solvents may be used. Water-insoluble fractions of 50-90% of ethanol extract or ethanol-soluble fraction of hot water extract can be used.

The extraction may be carried out by conventional methods such as hot water extraction, sonication, etc., and a lyophilized product of the extract can be used for the present composition. In addition, the extract can be further purified by conventional fractionation method or chromatography, and such fractionated material or purified material is also within the scope of the present invention. The present invention can be derived from different parts of bamboo (whole bamboo herb, branch, shell, leaf, sprout, root, endodermis, etc.,) the active ingredients include bamboo polyphenols, polysaccharides and shoot triterpenoids. A preferred embodiment of the extract of the invention includes a total phenol content of 8% to 30% polysaccharide content of 5% to 15%, triterpenes content of 3% to 10%—this provides an excellent natural antioxidant, immune enhancers, lipid metabolism regulators and quality improver. It can be used in the food industry to improve livestock and it can be used in humans as a fat reducing and weight loss supplement.

In particular embodiments a bamboo leaf extract which contains 1-99% by weight of bamboo leaf extract and comprising flavonoids and phenolic acids is provided wherein the total flavonoid content of bamboo leaf extract is 4-50% and the total phenolic acids content is 10-80%. The composition may also contain flavonoids including homoorientin, orientin, isovitexin and vitexin, and the phenolic acids comprise chlorogenic acid, ferulic acid, caffeic acid and p-coumaric acid. In particular embodiments, the phenolic acid composition is approximately as follow: total phenols: about 35.0 to about 61.0%; total flavonoids: about 12.0 to about 30.5%; chlorogenic acid: about 0.02 to about 2.0%; caffeic acid: about 0.02 to about 2.0%; p-coumaric acid: about 0.03 to about 3.5%; ferulic acid: about 0.01 to about 3.5%.

Another embodiment further comprises at least one extract selected from a group consisting of ginkgo extract, tea extract, rosemary extract, apple polyphenol extract, haw extract, onion extract, licorice extract, root of kudzu vine extract, grape seed extract and leech extract.

The composition of the present invention is appropriately administered depending on the extent of absorption of the active ingredients into the body; excretion rate; age, weight, sex, and condition of patient; severity of treated disease, etc. However, generally, the dosage for an adult is in solution 0.0001 to 100 mg/kg, or preferably 0.001 to 100 mg/kg, per day. It can be administered once a day or several times a day. The amount should not limit the scope of the present invention in any manner.

Complex supplements such as the bamboo extracts of the present invention may demonstrate non-linear, non-monotonic dose responses similar to low doses of endocrine disrupters act in ways that are totally unpredicted by the traditional approaches of toxicology. D. Fagan, Nature 490: 462 at 463 (25 Oct. 2012). This makes dosing and interpreting the results from studies of such complex products difficult. See also, Conolly, R. B. “Nonmonotonic Dose-Response Relationships: Mechanistic Basis, Kinetic Modeling, and Implications for Risk Assessment.” Toxicological Sciences 77.1 (2003): 151-57.

Extracts of bamboo according to the present invention may effectively inhibit fatty acid synthase. FAS will increase expression in the adipose tissue. Bamboo extracts according to the invention, comprising bamboo flavonoids, effectively inhibit FAS with an IC₅₀ value of about 0.60 μg/mL. The extracts push the balance between lipogenesis and energy generation forward to produce energy, and so reduce body weight. In addition, the activation of energy metabolism inhibits the expression of neuropeptide Y, promotes the expression of anorexia neuropeptides (such as alpha MSH, CART, and POMC) and indirectly suppresses appetite.

Extracts of bamboo according to the present invention may therefor be used in methods of promoting weight loss and improving general health, such as increased heart health, as demonstrated by improvement in the relevant measurements of cholesterol (Total cholesterol, high density lipoproteins (HDL or ‘good’ cholesterol) and low density lipoproteins (LDL or ‘bad’ cholesterol).

Additionally, extracts of bamboo according to the present invention may effectively increase the lean muscle mass in an animal. Such extracts may be incorporated into a composition that is useful for the treatment of muscle-wasting or muscle-weakness disorders, such as sarcopenia and dynapenia. Sarcopenia is highly prevalent in the elderly population, and may also occur with greater frequency in subjects with many other disease states and conditions, such including cancer cachexia, sepsis, denervation, disuse, acquired immunodeficiency syndrome (AIDS), chronic kidney or heart failure, unloading/microgravity, and muscle dystrophies. See Lynch et al. (2007) Pharcology & Therapeutics, 113: 461-487. Sarcopenia may be diagnosed by the presence of both low muscle mass and low muscle function (strength or performance). For example, muscle mass may be assessed using such criteria as measuring the mid-arm muscle circumference (MAMC); muscle strength may be assessed using handgrip dynamometer. See, Gariballa and Alessa (2013) Clinical Nutrition, 32:772-776. Increased lean muscle mass may be assessed by an increase in relevant biomarkers, such as creatinine.

In further embodiments, extracts of bamboo according to the present invention, and compositions containing such extracts, may be used for improving the nutritional quality and sensory qualities of feed for animals, especially poultry and other meat animals. Because of its effects on reducing fat and increasing lean muscle mass, inclusion of bamboo extract of the present invention in the diet of meat animals can increase the nutritional value and quality of meat from such animals. In particular embodiments, bamboo extract of the present invention is added to the diet of the meat animal in order to increase lean muscle mass, and/or reduce abdominal and/or subcutaneous fat thickness. Preferred species of meat animal include, for example, poultry, such as chicken, domestic duck, emu, ostrich, pheasant, quail, domestic turkey and goose; mammals, including bovines, such as cattle, bison, buffalo and domesticated yak; rabbits, pigs, sheep, deer, elk and goat; fish, such as yellow croaker, carp, haddock, cod, halibut, catfish, bass, tilapia, monkfish, snapper, swordfish, shark, salmon, and tuna; and other seafood animals including eel, squid, shrimp, and octopus.

In particular embodiments, Green Bamboo Extract (GBE) is added to feed for fish and exhibits favorable effects, including the reduction of body fat deposition and the improvement of nutritional quality and sensory qualities desirable for food, including taste, appearance, smell and texture.

In a particular embodiment, Green Bamboo Extract (GBE) is produced according to the methods of the present invention. The GBE comprises total phenols in an amount varying from about 8% to as much as about 30% of total weight of the extract; polysaccharide in an amount varying from about 5% to as much as about 15% of the total weight of the extract, and total triterpenoids in an amount varying from about 3% to as much as about 10% of the total weight of the extract. The GBE may be added to the basal diet of a meat animal, in an amount sufficient to provide an effective amount of phytosterols as antioxidants, which is generally in the amount of from about 1.5 g/kg to about 12.0 g/kg, preferably in an amount of from about 1.5 g/kg to about 6.0 g/kg.

The skilled artisan will recognize that many modifications, substitutions and additions to the invention as described above may be made without undue experimentation and would be expected to work in essentially the same manner and accomplish essentially the same result as described herein, without departing from the practice of the present invention. Hereinafter, the present invention will be described in more detail with reference to the following examples, but the scope of the present invention should not be construed to be limited thereby in any manner. All publications cited in the above description and in the examples below are hereby incorporated herein by reference.

### EXAMPLES

### Example 1

Dried bamboo (20 kg) may be extracted by adding 25% of ethanol (200 l) and heating the mixture at 80° C. for 6 hr. The extract is filtered and concentrated to remove the ethanol until the extract volume reaches 5 l. The concentrated extract may then be cooled to room temperature. The pellets are collected and dried to obtain the bamboo extract (approximately 790 g).

### Example 2

Dried bamboo (20 kg) may be extracted by subjecting it to three times of reflux extraction using purified water as solvent (1:5 ratio). The extract is filtered and concentrated under reduced pressure. Impurities are removed using centrifugal separation. The purified extraction is then subjected to a liquid-liquid extraction using n-butyl alcohol as solvent. Then the extract is further concentrated and dried to obtain bamboo extract (350 g).

### Example 3

Dried bamboo (20 kg) may be extracted by subjecting it to three times of reflux extraction using purified water as solvent (1:5 ratio). The extract is then absorbed with a macroporous resin. Washed with water and subsequently desorbed with ethanol as an eluent. Then the extract is further concentrated and dried to obtain bamboo extract. The yield is approximately 3%.

### Example 4

Dried bamboo may be extracted to a high purity by dissolving it in high hydrophilic organic solvent with concentrations appropriately varied; temperature might be increased to aid the process. Then impurities are removed using high-speed centrifugation or membrane separations. Next the extraction is passed through a liquid chromatography column pack with an adsorbent such as a macroporous resin and the like. The concentration can be dried to obtain bamboo extract.

### Example 5

Dried bamboo is dissolved on a mixture of distilled water and aliphatic components. Mixture is then filtered and concentrated at low pressure. Filtered mixture is feed into a distillation column where the mixture is fractioned and divided. Phytosterol rich portion is subsequently removed and dried into a powder to obtain bamboo extract.

### Example 6

### Bamboo Shoot Extraction and Fractionation

Dry bamboo shoot powder may be homogenized in distilled water for 30 minutes. The solution is then filtered through a vacuum filter with a coarse porosity (particle retention >10 um) filter paper. The water fraction is condensed in a rotary evaporator below 50° C. The water extraction (WE) filtrates may be stored at 4° C.

The remaining residues are further extracted with 100% ethanol for 4 hours. The extracts are condensed by rotary evaporation and slightly saponified with 50% KOH, refluxed for 30 min with moderate stirring in a water cooled reflux column at 75° C. This solution may be further extracted six times with petroleum ether. The petroleum ether extract is condensed by evaporation and dried under N₂. The crude saponified products (total crude fraction or TCE) may be further extracted with methanol and methylene chloride according to their polarity. After condensation in a rotary evaporator below 50° C. and drying under N₂ two semi-fractions are obtained: total methanol soluble fraction (TMS) and total methanol insoluble, i.e., methylene chloride soluble fraction (TMIS). Fractions TMS and TMIS may be further fractionated by reverse phase and normal phase column chromatography technologies, respectively. TMS may be further fractionated into multiple fractions and dried under N₂. It is not possible to fractionate and isolate TMIS further because the chemical structures of these compounds are too similar. Overall, 9 fractions may be obtained.

### Example 7

An inventive crude extract of bamboo is dried, cut, crushed and mixed with 5 to 25-fold, preferably, approximately 10 fold volume of distilled water, lower alcohols such as methanol, ethanol, butanol and the like, or the mixtures thereof, preferably methanol; the solution is treated with hot water at the temperature ranging from 20 to 100° C., preferably from 60 to 100° C., for the period ranging from 1 to 24 hours with extraction method by the extraction with hot water, cold water, reflux extraction, or ultra-sonication extraction with 1 to 5 times, preferably 2 to 3 times, consecutively; the residue is filtered to obtain the supernatant to be concentrated with rotary evaporator, at the temperature ranging from 20 to 100° C., preferably from 50 to 70° C. and then dried by vacuum freeze-drying, hot air-drying or spray drying to obtain dried crude extract powder which is soluble in water, lower alcohols, or the mixtures thereof.

Additionally, polar solvent soluble and non-polar solvent soluble extract of present invention can be prepared by the following procedure; the crude extract prepared as above is suspended in water, and then is mixed with 1 to 100-fold, preferably, 1 to 5-fold volume of non polar solvent such as ethyl acetate, chloroform, hexane and the like; the non-polar solvent soluble layer is collected to obtain non-polar solvent soluble extract of the present invention and remaining polar solvent soluble layer is collected to obtain polar solvent soluble extract of the present invention which is soluble in water, lower alcohols, or the mixtures thereof. Also, these procedures may be modified or subjected to further steps to fractionate or isolate more potent fractions or compounds by conventional procedure well-known in the art, for example, the procedure disclosed in the literature (Harborne J. B. Phytochemical methods: A guide to modern techniques of plant analysis, 3^{rd} Ed. pp 6-7, 1998).

### Example 8

### Extract of Bamboo Leaves and its Preparation

Dry bamboo leaf (14% moisture content) is crushed to about 10 mesh and subjected to purified water heat reflux extraction for 1.5 h (about 50 kg per 500 L), filtered and the resulting filtrate is concentrated under vacuum (Vacuum 0.09 Mpa, temperature of 55° C. to a solids content of about 25% (wt %), spray dried (inlet air temperature of 185° C., the air temperature 90° C., to yield 4.1 kg of extract (brown-yellow powder, total phenol content of 29.02% and 8.21% polysaccharides).

Canned boiled bamboo shoot processing byproducts may also be used as the starting material for this process.

### Example 9

### 50 Day Fat and Weight Loss Study on Chicken

Breeding Test Design

Three hundred and sixty healthy chickens at the age of 26-day and with the initial body weight of 375.12±8.30 g, were randomly selected into four groups (each group represented by three replicates of 30 chickens each). The control group was fed with basic daily ration (powdery mixtures), which was prepared referring to the Nutritional Requirements of Poultry 9^{th} ed. 1994, National Academy Press, Washington DC) poultry nutrition needs and divided into two stages (26-47 days age and 48-75 days age). Three test groups, with a 49 days feeding period were added 1.5, 3.0 and 6.0 g/kg green bamboo extract (GBE) into their basic daily ration, respectively.

All the chickens were fasting 12 h (self-help drinking water) when the feeding finished. Three chickens were randomly selected from each repeat, weighing and collecting wing vein blood. Centrifuged blood and collected the serum then sub-packaged and stored in a refrigerator at −20° C. till further use. The spleen, thymus, bursa of Fabricius, liver, abdominal fat were separated and weighted respectively after the chicken was slaughtered, the liver was stored in a refrigerator at −20° C. for further use. Divided the muscles from chest and leg, then part of chest muscle was used to measure the pH, drip loss, hardness, stickiness, flexibility and sensory evaluation, the other part of the chest and leg muscle were stored in a refrigerator at −20° C. for further use.

Effect of GBE on Lipid Metabolism and Body Fat of Chicken

The abdominal cavity is the main part of the chicken for accumulating body fat and so the percentage of abdomen fat and the thickness of subcutaneous fat reflect fat metabolism to a reasonable extent. In this experiment, the middle dose of 3.0 g/kg showed the largest decrease in abdominal fat while both the 1.5 and 3.0 g/kg doses induced an extremely significant decrease in the thickness of subcutaneous fat. Thus, adding the right dose of GBE to the daily ration of chickens can have dose responsive positive effects on reduction of body fat deposition, and effect on body weight.

TABLE 1
Effects of BBE on body fat deposition of meat chickens

| | GBE additive dose in daily ration (g/kg) | | | |
|---|---|---|---|---|
| Treatment | 0 | 1.5 | 3.0 | 6.0 |
| Weight/g | 1740.00 ± 103.41 | 1728.75 ± 73.85 | 1711.55 ± 119.19 | 1702.50 ± 79.45 |
| Percentage of | 3.73 ± 1.06^{a} | 2.85 ± 0.69^{bc} | 2.75 ± 0.65^{c} | 3.55 ± 0.96^{ab} |
| abdomen fat/% | | | | |
| Thickness of | 3.96 ± 1.16^{Aa} | 2.51 ± 0.33^{BCb} | 2.88 ± 0.58^{ACb} | 2.55 ± 1.33^{BCb} |
| subcutaneous fat/mm | | | | |

Shoulder letters in the same line denote the significance. Different small letters mean significant difference (p < 0.05), different capital letters mean highly significant difference(p < 0.01), the same letter or no letter means no significant difference (p > 0.05).

### Example 10

### Effects of GBE on Serum Leptin and Adiponectin of Chickens and the Correlation with Fat Deposition

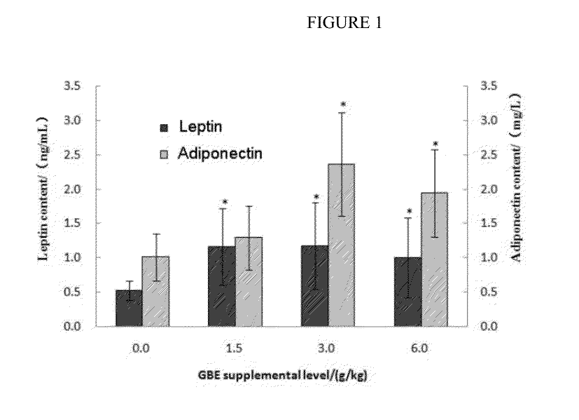

Leptin is a peptide hormone which synthesized and secreted from fat cells and has a variety of functions. As a negative feedback control signal, it participates in the negative regulation of body fat and plays a fundamental role in stabilizing the energy balance. Effects of GBE on leptin and adiponectin of meat chickens was demonstrated in FIG. 1. Leptin contents in chicken serum increased 123.08%, 125.00% and 92.31%, and adiponectin content increased 27.72%, 133.66% and 92.08%, followed by the GBE additive amount of 1.5, 3.0 and 6.0 g/kg, respectively. With the increase of additive amount, leptin level decreased after increased first, of which 3.0 g/kg additive amount group reached the highest level of leptin to a significant level. That means that the influence of GBE on the content of leptin in meat chicken serum has a two-way regulating role. The influence of the GBE on the adiponectin of leptin in meat chicken serum was the same as the leptin and the additive amount of 3.0 g/kg reached to significant level.

The addition of GBE to the diet of chickens thus had significant positive effects on lipid metabolism, reducing meat chicken abdominal fat, thickness of subcutaneous fat, liver fat content and serum triglyceride levels (p<0.05), improved unsaturated fatty acids in scale and the proportion of essential fatty acids in the chicken, and showed significant effects on serum leptin level, adiponectin content and antioxidant enzymes activity (p<0.05). Additionally, GBE-fed chickens scored higher on thymus index, bursal index and had higher serum lysozyme activity and significantly increased contents of serum immunoglobulin IgA and IgM (p<0.05). These results indicate that the addition of GBE as feed additive had significant effects in promoting immunity.

Additionally, addition of GBE to meat chicken feed resulted in significant effects on the improvement of chest muscle, fat content, adhesion, flexibility and chicken pH value after the chicken had been slaughtered for 45 min (p<0.05). GBE also decreased the drip loss rate and hardness of chest muscle (p=<0.05). Total essential contents, total amino acids and proportion of delicious and salty amino acids significantly improved, as well as the content of K, Ca, Mg, Fe, Zn, etc. (p=<0.05).

Because of the improved non-specific immune functions of GBE, it may be possible to reduce the use of antibiotics and other veterinary drugs in meat chickens fed diets supplemented with bamboo extracts. The positive effects of GBE on lipid metabolism, may help in producing low-fat meat chickens, with improved taste and nutritional value.

### Example 11

### Glucose Stabilization

It is expected for the described bamboo extract to improve fasting blood glucose levels, insulin resistance and reduce risk for diabetes in animals including humans. The previous example showed that the bamboo extract significantly improved adiponectin levels, a protein hormone that modulates glucose amongst other metabolic processes [see also Huang et al. Effects of Dietary Green Bamboo Extract on Lipid Metabolism of Broilers. Chinese Journal of Animal Nutrition, 2013, 25:148-155]. This hormone has been shown to suppress the metabolic derangements that may result in type 2 diabetes [Ukkola et al. “Adiponectin: a link between excess adiposity and associated comorbidities?”. J. Mol. Med. 80 (11): 696-702]. In that same study the bamboo extract was shown to significantly improve leptin levels. This in combination with the adiponectin has been shown to act synergistically, and completely reverse insulin resistance in mice. Yamauchi et al (August 2001). “The fat-derived hormone adiponectin reverses insulin resistance associated with both lipoatrophy and obesity”. Nat. Med. 7 (8): 941-6.

### Example 12

### Effects of Green Bamboo Extract on Muscle in Micropterus salmoides

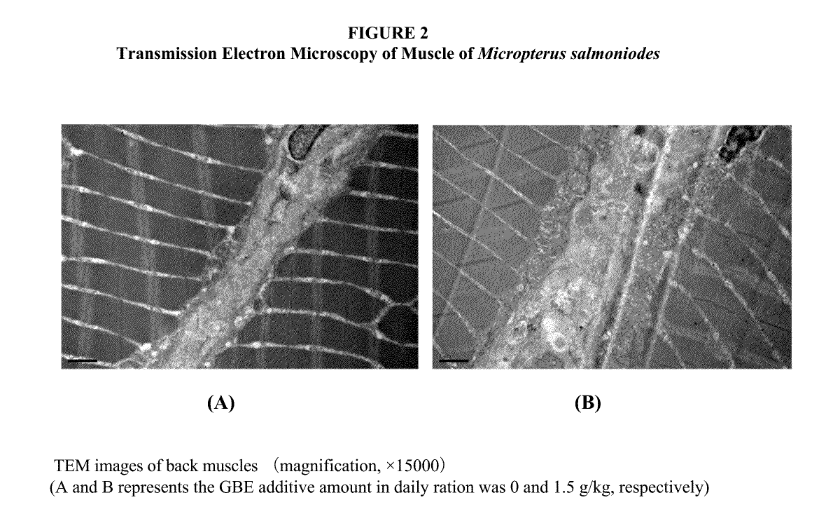

Fresh-water fish of the species Micropterus salmoides (also known as largemouth bass) are fed either a standard daily diet (control) or the same standard daily diet supplemented with 1.5 g/kg of green bamboo extract (GBE test group). Electron microscopy of the back muscles showed increases in the amount of mitochondria in the muscle of the GBE test group compared with the control group. See FIG. 2. Mitochondria are the primary energy centers of cells, and greater amounts of mitochondria in muscle correlate with a healthier more active fish. Additionally, greater energy may be useful for increasing lipid metabolism.

### Example 13

### Effects of Puffed Pellet Feed Supplemented with Green Bamboo Extract (GBE) on Lipid Metabolism and Edible Quality of Pseudosciaena crocea

METHODS and MATERIALS:

Yellow croaker fish (Pseudosciaena crocea) are an important marine economic fish in China. One thousand and eight hundred of large yellow croakers with the average weight of 150.06±11.35 g, were randomly selected into three groups (each group represented by three replicates of 200 large yellow croakers each). The GBE group and astaxanthin group were feed with complete formula puffed pellet feed rich in 3.0 g/kg of GBE and 1.0 g/kg of astaxanthin, respectively. Astaxanthin is an accepted dietary additive, but is quite expensive. The reference group was feed with chilled miscellaneous fish and shrimp. The breeding time was five months. Feeding was stopped 24 h after the test. Eight test large yellow croakers were randomly selected from each replicate group (that is, 24 test large yellow croakers each group). The selected fish were weighed, the livers were segmented, and the abdominal and back muscles examined after they were killed. Adequate amount of fresh back and abdominal muscle were used for sensory evaluation, the rest were sub-packaged and stored on −20° C. refrigerator for further use. The fat content of liver and fish and the superoxide dismutase (SOD) activity, catalase (CAT) activity, malondialdehyde (MDA) content, leptin content, adiponectin content of liver were detected. The back muscle tissue was observed with transmission electron microscope.

Results

Effect on Body Weight and Fat Content of Different Parts of Large Yellow Croaker

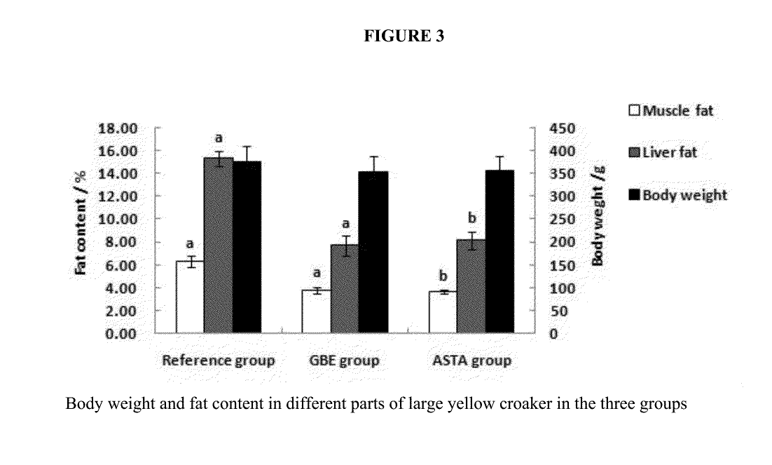

The body weight and fat content in different parts of large yellow croakers in the three groups are shown in FIG. 3. Overall body weight in the three groups exhibited no significant differences. However, the fat content of liver and muscle in the GBE group and the astaxanthin group was significantly lower than the reference group (p<0.05); there was no significant difference between GBE group and astaxanthin group (p>0.05).

Effect on Leptin and Adiponectin Levels in the Liver of Large Yellow Croaker

Fat content in the livers of large yellow croaker was significantly higher than that in muscle. The fat content in livers of the three groups also showed significant differences from each other. Therefore, further research on liver fat of large yellow croaker and the related metabolic factors leptin and adiponectin was performed. The levels of related metabolic factors (leptin and adiponectin) in the liver of large yellow croaker was measured and is shown in Table 2. The leptin content in the liver of GBE group was significantly higher than reference group and astaxanthin group (p<0.05), while the adiponectin level in the liver of GBE group was significantly higher than reference group (p<0.05), and lower than astaxanthin group, but not by a statistically significant difference (p>0.05).

TABLE 2
Leptin and adiponectin levels in the liver
of large yellow croaker in the three groups

| | REFERENCE | GBE | ASTA |
|---|---|---|---|
| | GROUP | GROUP | GROUP |
| LEPTIN (ng/mL) | 0.89 ± 0.21^{a} | 1.59 ± 0.32^{b} | 1.08 ± 0.12^{b} |
| ADIPONECTIN | 0.74 ± 0.07^{a} | 1.80 ± 0.15^{b} | 2.57 ± 0.33^{c} |
| (ug/mL) | | | |

Note:
in the same row, values with different letter superscripts mean significant difference (p < 0.05), and with the same letter or no letter superscripts mean no significant difference (p > 0.05).

Effect on Activities of Antioxidant Enzymes and Lipid Peroxidation in Liver of Large Yellow Croaker

The activities of antioxidant enzymes SOD, CAT, and the content of lipid peroxidative product CAT in the liver of large yellow croakers were measured and shown in Table 3. The activities of SOD, CAT, ratio of CAT to SOD in the liver of GBE group and astaxanthin group were obviously lower than reference group (p<0.05). the MDA content in the liver of GBE group was evidently lower than reference group and astaxanthin group (p<0.05).

TABLE 3
Activities of SOD, CAT, ratio of CAT to SOD and level of MDA
in the liver of large yellow croaker in the three groups

| | REFERENCE | GBE | ASTA |
|---|---|---|---|
| | GROUP | GROUP | GROUP |
| SOD/(U/mg | 264.33 ± 12.12^{b} | 167.29 ± 20.33^{a} | 157.35 ± 10.24^{a} |
| prot) | | | |
| CAT/(U/mg | 67.56 ± 11.43^{b} | 23.24 ± 5.14^{a} | 22.56 ± 3.11^{a} |
| prot) | | | |
| CAT/SOD | 0.26 ± 0.01^{b} | 0.14 ± 0.01^{a} | 0.13 ± 0.01^{a} |
| MDA/(mmol/ | 257.64 ± 21.15^{c} | 52.39 ± 9.44^{a} | 117.65 ± 9.33^{b} |
| mg prot) | | | |

Note:
in the same row, values with different letter superscripts mean significant difference (p < 0.05), and with the same letter or no letter superscripts mean no significant difference (p > 0.05).

Effect on Microstructure of Back Muscle of Large Yellow Croakers

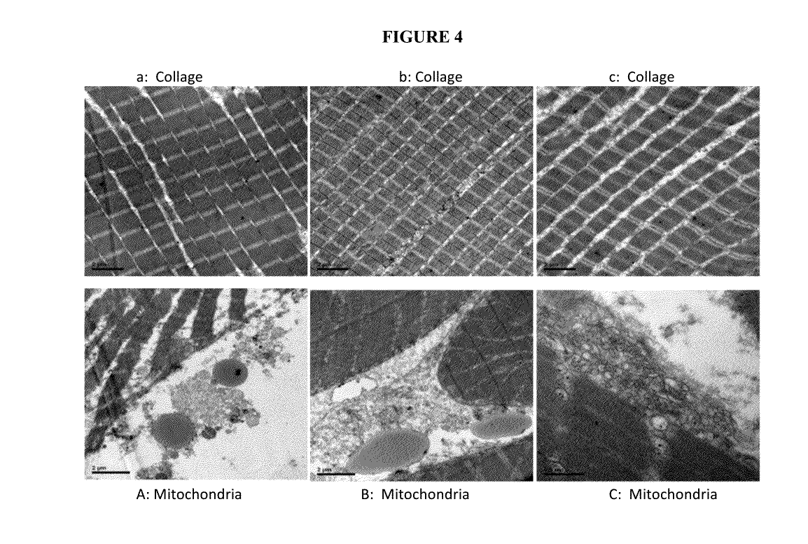

The transmission electron microscopic images of back muscles of large yellow croakers in the three groups are shown in FIG. 4. Observed from the 10000 times photo, we could find that the clearance of muscle bundle share the order as: reference group<GBE group<astaxanthin group. Observed from the 12000 times photo, we could find that the quantity of mitochondria share the order as: reference group<GBE group<astaxanthin group.

Effects on Sensory Qualities

Samples from each of the Reference Group, the GPE Group and the ASTA group were subject to sensory evaluation, conducted by a group of eight specially trained individuals. Boiled fish fillet was described as fish fillet (40 g) after been boiled in 400 mL of capped boiling purified water for 5 min then opened the cap and evaluated. The weight of each indicator was set as: the color, odor, muscle texture and elasticity of raw fish fillet were 0.10, 0.10, 0.15 and 0.10, respectively, the odor, taste and soup form of boiled fish fillet were 0.20, 0.25 and 0.10, respectively. Statistical of data was using fuzzy mathematics. The average score multiplied by the weight of each indicator became the indicator score. The summation of seven indicators of raw fish fillet and boiled fish fillet was defined as for sensory evaluation scores. The standard for evaluation are listed in Table 4:

TABLE 4
Sensory Quality Evaluation Scale

| | | Very good | Good | Medium | Poor | Very poor |
|---|---|---|---|---|---|---|
| Item | Description | (5 points) | (4 points) | (3 points) | (2 points) | (1 point) |
| Raw | Color | Normal color, | Normal color, | Little dim color, | Little dim color, | Dim color, the |
| Fish Fillet | | the muscle | the muscle | the muscle | the muscle | muscle section |
| | | section is greatly | section is shiny. | section is | section is | is reluster |
| | | shiny. | | slightly shiny. | reluster | |
| | Odor | The inherent | The inherent | The inherent | The inherent | With strong |
| | | fragrance is | fragrance is less | fragrance is | fragrance is | stench or |
| | | strong. | strong. | light, with little | disappeared, | ammonia smell. |
| | | | | off-flavor. | with stench or | |
| | | | | | ammonia smell. | |
| | Muscle Texture | The muscle | The muscle | The muscle | The muscle | The muscle |
| | | tissue is | tissue is | tissue is not | tissue is not | tissue is not |
| | | compact and | compact, the | compact and | compact but | compact and |
| | | complete, the | texture is clear. | not loose. | partly loose. | loose. |
| | | texture is very | | | | |
| | | clear. | | | | |
| | Muscle | Solid and highly | Solid and elastic, | Elastic, the | A little elastic, | No elasticity, |
| | Elasticity | elastic, the | the sunken | sunken | the sunken | the sunken not |
| | | sunken | disappeared | disappeared | disappeared | disappeared |
| | | disappeared | after depression | slowly after | quite slowly | after depression |
| | | immediately | finger pressure | depression | after depression | finger pressure |
| | | after depression | relaxed. | finger pressure | finger pressure | relaxed |
| | | finger pressure | | relaxed. | relaxed | |
| | | relaxed. | | | | |
| Boiled | Odor | The inherent | The inherent | The inherent | The inherent | With strong |
| Fish Fillet | | fragrance is | fragrance is little | fragrance is | fragrance is | stench or |
| | | freshness. | freshness. | light, with little | disappeared, | ammonia smell. |
| | | | | off-flavor. | with stench or | |
| | | | | | ammonia smell. | |
| | Taste | The inherent | The inherent | The inherent | No umami, no | No umami, the |
| | | umami is strong, | umami is less | umami is light, | off-flavor, the | meat is |
| | | the elasticity of | strong, the meat | the elasticity of | elasticity of | anabrotic, not |
| | | meat is good. | is elastic. | meat is normal. | meat is weak. | elastic and with |
| | | | | | | ammonia smell. |
| | Soup form | Very clear, no | Clear, no meat | A little clear, | The meat | The meat is |
| | | meat in the | in the soup | little meat in the | suspend in soup, | corrupt and |
| | | soup | | soup | the soup is | suspend in soup, |
| | | | | | muddy. | the soup is |
| | | | | | | muddy. |

TABLE 5
Results of the Sensory Evaluation
The results of sensory evaluation of large yellow croakers in the
three groups are shown in Table 5. Statistical results of fuzzy
mathematics show that the color, odor, muscle texture and muscle
elasticity of raw fish fillet of GBE Group were superior to the
Reference Group and close to the Astaxanthin Group. All indicators
scores of boiled fish fillet of the GBE Group were significantly
higher than the Reference Group and close to the Astaxanthin Group. With
respect to, especially taste, the GBE Group received the highest score.

| | | Reference | GBE | ASTA |
|---|---|---|---|---|
| Item | Description | Group | Group | Group |
| Raw | Color | 3.3 ± 0.3 | 3.8 ± 0.2 | 3.8 ± 0.4 |
| Fish | Odor | 3.4 ± 0.3 | 3.7 ± 0.2 | 3.7 ± 0.3 |
| Fillet | Muscle | 3.2 ± 0.2^{a} | 4.1 ± 0.5^{b} | 4.2 ± 0.3^{b} |
| | Texture | | | |
| | Muscle | 3.0 ± 0.4 | 3.2 ± 0.2 | 3.4 ± 0.2 |
| | Elasticity | | | |
| Boiled | Odor | 2.9 ± 0.2^{a} | 3.8 ± 0.2^{b} | 3.9 ± 0.4^{b} |
| Fish | Taste | 2.6 ± 0.3^{a} | 4.0 ± 0.2^{b} | 3.8 ± 0.5^{b} |
| Fillet | Soup form | 3.0 ± 0.1^{a} | 3.8 ± 0.2^{b} | 3.8 ± 0.4^{b} |

| | | | |
|---|---|---|---|
| Total Score | 21.4 ± 2.1^{a} | 26.3 ± 2.4^{b} | 26.6 ± 2.5^{b} |

Note:
in the same row, values with different letter superscripts mean significant difference (p < 0.05), and with the same letter or no letter superscripts mean no significant difference (p > 0.05).

Conclusions

Compared with the reference group, large yellow croakers in GBE group had significant lower fat content in muscle and liver (p<0.05), larger muscle bundle gap and more mitochondria in back muscles, significantly higher levels of leptin and adiponectin in liver (p<0.05); significantly lower levels of SOD, CAT, CAT/SOD and MDA in liver (p<0.05); the tested results of large yellow croakers in GBE group and ASTA group were similar. In conclusion, puffed pellet feed supplemented with GBE could significantly regulate the lipid metabolism of large yellow croaker, reduce its body fat deposition and improve its edible quality. The effects of GBE (at the dose of 3 g/kg) were close to that of ASTA (at the dose of 1 g/kg).

### Example 14

### Human Weight Loss Study

Methods and Materials:

Two subjects of different age (between ages 18 to 65) and ethnic background were dosed with three capsules, twice a day, of bamboo extract, standardized to provide a daily intake of 750 mg of bamboo antioxidants. Subjects were instructed to add exercise and a 2000 calorie daily diet, and were subsequently followed for eight weeks. Subjects were measured at the beginning and end of the study period. In addition to weight, heart health biomarkers such as cholesterol (i.e., total, HDL, LDL and triglycerides), general chemistry and electrolytes were measured as well as hematology, including glycosylated hemoglobin.

Results:

Results of the study are shown in Tables 2 and 3. Subjects lost an average of 1.35 lbs over the 8 week study with a gross BMI reduction of 1% and 1.5 inch reduction in waist circumference. Additionally, subjects exhibited an overall improvement in cholesterol, with a clinically relevant 31% reduction of total cholesterol (TC), a 22% increase in HDL cholesterol (sometimes referred to as ‘good’ cholesterol), and a 42% decrease in LDL cholesterol (sometimes referred to as ‘bad’ cholesterol). With all cholesterol markers at normal levels after the intervention from an average of 206.5, 54, 122 to an average of 175, 66, 70 mg/dL for TC, HDL, and LDL, respectively. There was also a general improvement in all other measured chemical biomarkers. Sodium decreased by an average of 3.2%; Potassium by 10.7%; Chloride by 5.7%; Fasting Glucose by 17%; Blood Urea Nitrogen (BUN) by 18.9%; and Anion Gap by 14.7%. Other parameters that showed clinically significant improvements included increased ionized Calcium, by an average increase of 20.6%; creatinine increase of 28% (significant indication of lean muscle increase).

TABLE 4
CHOLESTEROL PROFILE

| | AVG @ | AVG @ | |
|---|---|---|---|
| | VISIT 1 | VISIT 2 | % CHANGE |
| Total Cholesterol | 206.5 | 175 | −31.5 |
| (mg/dL) | | | |
| HDL (mg/dL) | 54 | 66 | +12 |
| LDL (mg/dL) | 122.5 | 70 | −52.5 |

TABLE 5
CHEMICAL PROFILE

| | AVG @ | AVG @ | |
|---|---|---|---|
| | VISIT 1 | VISIT 2 | % CHANGE |
| Sodium (mmol/L) | 141.5 | 137 | −3.2 |
| Potassium (mmol/L) | 4.2 | 3.75 | −10.7 |
| Chloride (mmol/L) | 105.5 | 99.5 | −5.7 |
| Ionized Calcium (mmol/L) | 1.02 | 1.23 | +20.6 |
| TCO2 (mmol/L) | 24.5 | 28.5 | +16.3 |
| Glucose (mg/dL) | 117.5 | 97.5 | −17.0 |
| Blood Urea Nitrogen | 18.5 | 15 | −18.9 |
| (mg/dL) | | | |
| Creatinine (mg/dL) | 1.25 | 1.6 | +28.0 |
| Hct (% PCV) | 55 | 56.5 | +2.7 |
| Hb (g/dL) | 18.7 | 19.2 | +2.7 |
| Anion Gap (mmol/L) | 17 | 14.5 | −14.7 |

Accordingly, human subjects fed a diet supplemented with bamboo extract exhibited improvements in health indicia such as weight loss and reduction in BMI, with significant improvements in cardiac disease markers, such as cholesterol level (i.e. LDL, HDL, triglycerides etc.); general chemical and electrolyte markers (i.e. sodium, potassium, chloride, TCO2, Anion Gap, Ionized Calcium, Glucose, Urea Nitrogen, Creatine) and hematology including glycosylated hemoglobin.

The bamboo extract of the invention was effective to reduce the weight of the participants, increase serum leptin and adiponectin, improve cholesterol status, and reduce fat, while maintaining or improving healthy chemical and electrolyte status.

1-21. (canceled)

22. A composition for increasing fat metabolism in an animal comprising an extract of bamboo.

23. A composition for reducing the body weight of an animal comprising an extract of bamboo.

24. A method of increasing the fat metabolism of an animal by supplying the animal with the composition of claim 22.

25. A method of reducing the body weight of an animal comprising supplying the animal with the composition of claim 23.

26. A composition for animal feed comprising a phytosterol-containing extract isolated from bamboo.

27. The composition of claim 26, wherein said composition is prepared for addition to animal feed compositions.

28. A method of increasing the lean muscle mass of an animal comprising supplying the animal with the composition of claim 22.

29. A method of decreasing the abdominal fat of an animal by supplying the animal with an effective amount of the composition of claim 22.

30. A method of improving the food quality of an animal comprising feeding the composition of claim 26 to the animal.

31. A method of improving the food quality of an animal comprising adding the composition of claim 27 to the daily diet of the animal.

32. The method of claim 30, wherein said animal is selected from the group consisting of poultry, livestock and fish.

33. The method of claim 31, wherein said animal is selected from the group consisting of poultry, livestock and fish.

34. A method of improving the sensory quality of an animal comprising adding the composition of claim 26 to the daily diet of the animal.

35. A method of improving the sensory quality of an animal comprising feeding the composition of claim 27 to the animal.

36. The method of claim 34, wherein the meat of the animal is improved in at least one sensory quality selected from the group consisting of taste, appearance, smell and texture.

37. The method of claim 35, wherein the meat of the animal is improved in at least one sensory quality selected from the group consisting of taste, appearance, smell and texture.

38. A method of improving endurance in a human subject, comprising administering an effective amount of the composition according to claim 26 to the human subject, that is sufficient to improve the muscle's cellular composition.

39. A method of increasing improving cellular composition and energy in a human subject comprising administering an effective amount of the composition according to claim 26 that is sufficient to increase concentration and/or improve structure of mitochondria.

40. A method for treating a subject with a muscle-wasting disease comprising administering an effective amount of the composition of claim 26 to the subject.

41. The method of claim 40, wherein said muscle-wasting disease is selected from the group consisting of sarcopenia and dynapenia.
